(19) United States
(12) Patent Application Publication
(10) Pub. No.: US 2010/0048451 A1
(43) Pub. Date: Feb. 25, 2010

(54) NOVEL SPILL-RESISTANT FORMULATIONS COMPRISING HYDROCOLLOIDAL POLYMERS

(75) Inventors: Satish Asotra, Brampton (CA); Zoltan Bodor, Pickering (CA); Xiaopin Jin, Mississauga (CA)

Correspondence Address: TARO PHARMACEUTICALS U.S.A., INC.;C/O VENABLE LLP, P.O. BOX 34385, WASHINGTON, DC, 20043-9998, US

(73) Assignee: Taro Pharmaceuticals North America, Inc., Grand Cayman (KY)

(21) Appl. No.: 12/162,906

(22) PCT Filed: Apr. 6, 2007
(86) PCT No.: PCT/US07/08587

§ 371 Date: Sep. 30, 2009

Related U.S. Application Data
(60) Provisional application No. 60/744,368, filed on Apr. 6, 2006.

(51) Int. Cl. A61K 47/36; A61K 47/00; A61K 38/02; A61K 31/7088; A61K 31/137; A61K 31/57

(52) U.S. Cl. 514/2; 514/782; 514/777; 514/44 R; 514/653; 514/179

(57) ABSTRACT

The invention relates to novel spill resistant formulations comprising two or more hydrocolloidal polymers and an effective amount of a pharmaceutically active agent. The formulations have a viscometric yield value of a semi-solid, and a pH and spill-resistant consistency allowing for easier preparation and use of the pharmaceutical composition.

### BACKGROUND OF THE INVENTION

Syrups, elixirs, solutions, and suspensions are traditional dosage forms for oral delivery of medication. These liquid formulations are typically measured by pouring into a spoon or cup, but inadequate filling or spilling from the spoon may result in inaccurate dosage. Loss of motor skills or poor attention due to age or infirmity can cause difficulty in filling a spoon with a liquid and bringing it to the mouth. This may be a serious impediment to administering medicine. Solid formulations such as pills, tablets, and capsules are difficult for children, the elderly and the infirm to swallow.

Spill-resistant preparations for the oral delivery of pharmaceutics have been described in the commonly owned U.S. Pat. Nos. 5,881,926, 6,071,523, 6,102,254, 6,355,258, and 6,399,079, herein incorporated by reference. These patents define spill-resistant pharmaceutical preparations by physical parameters. The parameters include: (i) viscosity; (ii) ease of administration; (iii) mutual compatibility of the components of the formulation; and, (iv) shelf-life stability. Ease of administration is defined by extrudability, spreadability, and spill-resistance. The formulations comprise an active ingredient, a vehicle, and a thickening agent. The thickening agents may be a cellulose derivative or carboxyvinyl polymers. A preferred carboxyvinyl polymer is carbomer.

Carbomer gels exhibit maximum viscosity at about neutral pH, where the viscosity plateaus between pH values of 6.3 to 7.0. This pH-viscosity interaction of the carbomer polymer has restricted the use of weak acids and weak bases in spill-resistant pharmaceutical formulations. At higher or lower pH ranges, the ranges where the weak acids and bases are most soluble, the formulations lose their spill-resistant properties.

Hydrocolloid polymers such as the alginates and gums are known to absorb water and act as gelling agents and thickening agents in pharmaceutical compositions. U.S. Pat. No. 5,288,479 describes an extrudable oral gel composition consisting of pharmaceutical composition comprising a pharmaceutically active ingredient and a seaweed polysaccharide. The seaweed polysaccharides provides for an elastic and rheologically solid composition.

U.S. Pat. No. 6,071,523, demonstrate that not all thickening agents provide for a spill-resistant formulation. In the examples carrageenan, agar, a variety of cellulose derivatives and even honey, were tested for spill-resistance. The results demonstrate that merely adding a hydrocolloid polymer to a pharmaceutical base does not result in the physical properties that have been described as spill-resistant characteristics.

There is a need for novel spill-resistant formulations that incorporate active ingredients that may not be stable at neutral pH.

### SUMMARY OF THE INVENTION

The invention provides for a pharmaceutical composition having two or more hydrocolloidal polymers, an effective amount of a pharmaceutically active agent, and a viscometric yield value of a semi-solid. The composition has a spill-resistant consistency permitting the formulation to be squeezed by light manual pressure through a channel of about 1 to about 5 mm, to spread in a spoon bowl sufficiently quickly for accurate measurement, and to remain in the spoon bowl without spilling for at least about one second and less than about 20 seconds on spoon tilting and for at least about 30 seconds upon spoon vibration.

The hydrocolloidal polymers of the present invention may be selected from the group consisting of carrageenans, alginates, propylene glycol alginate, xanthan gum, guar gum, locust bean gum, tragacanth, gum karaya, gum ghatti, gum arabic, agar-agar, konjac glucomannan, pectin, tara gum, gellan gum, pullulan, curdlan, gelatin, chitosan, fenugreek gum and combination mixtures thereof. These gums are useful in oral medications and food applications as thickeners.

A pharmaceutically active ingredient of the present invention may be selected from the group consisting of analgesics, anti-inflammatories, antipyretics, antibiotics, antimicrobials, laxatives, anorexics, antihistamines, antiasthmatics, antidiuretics, antiflatuents, antimigraine agents, antispasmodics, sedatives, antihyperactives, antihypertensives, tranquilizers, decongestants, beta blockers; peptides, proteins, oligonucleotides and other substances of biological origin, and combinations thereof. More specifically the pharmaceutically active agent of the present invention may be selected from the group consisting of acyclovir, atenolol, atropine, ciprofloxacin, dilitiazem, diphenhydramine, diphenhydramine HCI, epinephrine, azithromycin, clarithromycin, guaifenesin, ephedrine, glucosamine, glucosamine sulfate, hydrochlorothiazide, metoprolol, nortriptyline, phenytoin, propoxyphene, propranolol, terfenadine, tetracycline, pseudoephedrine, captopril, diclofenac, enalapril, furosemide, ketoprofen, phenobarbital, naproxen, ibuprofen, lovstatin, penicillin G, piroxicam and ranitidine and mixtures and salts thereof.

More particularly, the invention is for a pharmaceutical composition comprising two or more hydrocolloidal polymers and an effective amount of a pharmaceutically active agent; having a viscometric yield value of a semi-solid; and a spill-resistant consistency permitting the formulation to be squeeze by light manual pressure through a channel of about 1 to about 5 mm to spread in a spoon bowl sufficiently quickly for accurate measurement, and to remain in the spoon bowl without spilling for at least about one second and less than about 20 seconds on spoon tilting and for at least about 30 seconds upon spoon vibration wherein the composition is adjusted to an can be adjusted to a pH of between about 3.5 and about 9.0.

### DETAILED DESCRIPTION

The invention relates to a pharmaceutical formulation for oral administration, comprising an effective amount of particles of a water-soluble or water insoluble active ingredient in a pharmaceutically acceptable aqueous vehicle.

The term spill-resistant formulation refers hereto a product which, as sold, has viscosity in a certain range, is a semi-solid, is easy to administer accurately, is storage stable, and has mutually compatible ingredients, as described in U.S. Pat. No. 6,071,523 to Mehta, herein incorporated by reference.

Generally the formulation has a viscometric yield value determined as a relative value, e.g. using a Brookfield Viscometer to measure a shear vs. stress curve. Ease of administration is intended to mean (a) extrudability under light manual pressure from a squeezable container or a proxy (e.g. a syringe with a 5 mm orifice), and (b) spreadability in a spoon bowl measured by extruding the formulation into a spoon bowl and determining whether the material levels or spreads to the edges of the spoon bowl. Spreadability also contributes to accuracy of measurement. The spill-resistant formulation according to the invention begins to spill from a spoon bowl during test periods of vibrations, inversion, and tilting, quickly enough to enable the product to be readily consumed from a spoon bowl by a patient but slowly enough to remain in the spoon, without spilling, in the time between dispensing and ingesting.

The formulation may have a Brookfield viscosity within the range of about 5,000 cps to about 17,500 cps at room temperature. Viscosity can be measured using a Brookfield Viscometer with a ‘T-C’ spindle at 20 RPM and 20-25 degrees Centigrade, or equivalent. Formulations exhibit desirable spill-resistant properties at a viscosity greater than about 5,000 cps. The product spreads at viscosity less than about 17,500 cps. The viscosity of the formulation increases with a decrease in temperature and decreases with increase in temperatures. However, these changes in the viscosity and correlated spill-resistant characteristics are reversible, so that the original formula viscosity is obtained when temperature returns to room temperature (˜23° C.; broadly 19° C. to about 29° C.). The formulation may be squeezed into a spoon from a container with light manual pressure; spread and level in a spoon bowl quickly enough for accurate measurement (typically in about 1-5 seconds at room temperature); and remain in the spoon bowl long enough to permit administration without spilling particularly under difficult circumstances such as encountered with dispensing to children, or by the elderly.

In addition to the tests provided for in the prior art, we have found that the consistency of a sample as measured by its resistance to flow under specific conditions is useful for screening for spill-resistant formulations. The Bostwick Consistometer (CSC Scientific Company Inc., Fairfax, Va.) is one of many instruments designed to make such measurements. The consistometer is a device with a slight gradient and a gate, which closes to form a compartment in which the sample can be placed. The test consists of measuring the distance covered by a given sample of fluid over a flat slot in a conventional time interval and at a constant temperature. This simple test is used widely to measure the variability of viscosity and to quantify non-Newtonian viscous behavior. The spill-resistant formulation of the invention is characterized for flow characteristics using a consistometer. After a sample of given weight is dispensed into the holding hopper a spring-loaded gate is opened. The distance the sample moves in the graduated trough is observed at suitably selected time intervals (example: 15 and 30 seconds). It has been found that the preferred spill-resistant characteristics of the invention correspond to observed flow rates of between about 10 to 20 cm/minute at 15 seconds and have a rate of about 5 to 10 cm/minute at 30 seconds.

The spill-resistant formulations have an observed flow rate on the Botswick consistometer. The observed flow rate of the composition in the consistometer at the 15 second time is about 10 to 20 cm/minute and at 30 seconds has a rate of about 5 to 10 cm/minute. Preferably, the viscosity of the present invention is between about 6,000 to about 15,500 cps, more preferably from about 7,000 to about 13,000 cps, most preferably from about 7,750 to about 12,000 cps.

Spill-resistance refers to the product's ability to withstand a series of tests that were developed to evaluate the product's spill-resistance. For most formulations, spill-resistance means the formulation does not spill from a teaspoon for a definite period, e.g. at least about 30 or 60 seconds on spoon inversion, about 30 or 60 seconds on spoon vibration, and about 10, 20, or 30 seconds on spoon tilting. Spill-resistant properties correlate with viscosity but are not necessarily directly linked, so that a composition within the target viscosity range may lack spill-resistance. The shaking, tilting and inversion tests are performed on an experimental platform as described in U.S. Pat. No. 6,071,523. Spill-resistance is related to whether the formulation passes a flow test, ensuring that dispensing and dosing to a 5.0 mL teaspoon is easy and satisfactorily accurate.

The inventive compositions have a homogeneity wherein the ingredient is uniformly dispersed or dissolved in the vehicle. The compositions have crystalline stability such that the active ingredient does not exhibit excessive crystalline growth or dissolution, so that the particles stay within a target particle size range or solubilized. Heat-cool studies can be conducted to analyze for crystal growth and active dissolution. The suspended formulations have a stability such that the active ingredient remains indefinitely without agitation, that is without stirring or shaking. This uniformity of the suspension allows for consistent dosing and an increased shelf life of the product, as the active ingredient remains uniform per dose administered. A semi-solid formulation of the invention can not be shaken easily, so the active agent must remain suspended or in solution without shaking. Advantageously, there is no need to shake the inventive compositions.

Exemplary of the active components which may be suitable for use in the present dosage forms may include, without limitation, analgesics, anti-inflammatories, antipyretics, antibiotics, antimicrobials, laxatives, anorexics, antihistamines, antiasthmatics, antidiuretics, antiflatuents, antimigraine agents, antispasmodics, sedatives, antihyperactives, antihypertensives, tranquilizers, decongestants, beta blockers; peptides, proteins, oligonucleotides and other substances of biological origin, and combinations thereof. Some examples of active pharmaceutical ingredients that may be used in the formulation are, for example, acyclovir, atenolol, atropine, ciprofloxacin, dilitiazem, diphenhydramine, diphenhydramine HCl, epinephrine, azithromycin, clarithromycin, guaifenesin, ephedrine, glucosamine, glucosamine sulfate, hydrochlorothiazide, metoprolol, nortriptyline, phenytoin, propoxyphene, propranolol, terfenadine, tetracycline, pseudoephedrine, captopril, diclofenac, enalapril, furosemide, ketoprofen, phenobarbital, naproxen, ibuprofen, lovstatin, penicillin G, piroxicam, ranitidine and mixtures and salts thereof.

In addition to the active ingredient, the inventive formulation comprises two or more hydrocolloidal polymers. These polymers are colloidally dispersible and soluble in water. Hydrocolloids are mainly long-chain, straight or branched polysaccharides that contain hydroxyl groups than can bond to water molecules. These chains can consist of 2,000 to over 10,000 monosaccharide units. The sugar monomers can contain linked side units, or substituent groups, such as sulfates, methyl ethers, esters and acetals. When added to water, these polymers form gels. Polymers that are hydrocolloidal are both organic and non-organic, naturally occurring and synthetic. Some examples of hydrocolloids are alginates, carrageenan gum, tragacanth gum, xanthan gum, and locust bean gum and esters and salts thereof. Alginic acid, sodium alginate, propylene glycol alginate, calcium alginate are esters and salts of alginate. Locust bean gum is a nonionic, neutral polysaccharide that is relatively unaffected over a wide range of pH. Hydrocolloids are known to have synergistic interactions, allowing for lower amounts of individual components to be used. However, care must be used in selecting the combination of hydrocolloids because irreversible binding and precipitation of the combination may result.

The inventive solution or suspension also comprises a vehicle. Propylene glycol, sorbitol and glycerin are widely used as a solvents, extractants, and preservatives in a variety of pharmaceutical formulations. The vehicle component may comprise up to about 50% propylene glycol, up to about 50% sorbitol and up to about 70% glycerin. The propylene glycol, glycerin and sorbitol further improve the flow of the formulation at the higher viscosities, and maintain the desirable spill-resistant properties. Purified water makes up the bulk of the carrier component of the formulation.

In addition to the thickening properties, the gums are stable over broad ranges of pH. For instance, mixtures of tragacanth and locust bean gum have shown to impart yield value and spill-resistant properties to some of the inventive formulations. In an aqueous solution gum tragacanth are known to maintain stable viscosity over a broad range. The acceptable range of pH for a stable viscous solution of gum tragacanth may be from a pH of about 2 to a pH of about 8. A solution comprising a combination of tragacanth with glycerin and locust bean gum, provided for a solution that had a viscosity and shear rate of a spill-resistant formulation. The ratio of tragacanth to locust bean gum was about 1 to 10 to about 1 to 5, and glycerin may be up to 50% of the formulation. The pH can be between about 2 to about 8, more preferably from 4 to about 6.

Another embodiment of the invention is the present invention is the use of three hydrocolloidal polymers with an aqueous base comprising water and non-aqueous vehicles. The non-aqueous vehicles may be selected from the group consisting of propylene glycol, glycerin and sorbitol. The combination of carrageenan, locust bean gum and xanthan gum gives an acceptable spill resistant formulation. In particular, we have found carrageenan: locust bean gum: xanthan gum in a ratio of about 3:1:5, more particularly, 6:1:2.3 gave an acceptable formulation. In particular, the formulation is tolerant of an active pharmaceutical ingredient having an acidic radical, the pH range of the formulation can be maintained between 3.0 to 5.0, and the rheology of the formulation is positive on the spill-resistant standards.

The inventive formulations may have an antimicrobial activity satisfying microbial challenge requirements such as USP, either due to preservatives or a low water activity (about 0.752 to about 0.838). Benzoic acid, sodium benzoate, sorbic acid, potassium sorbate, propionic acid, sodium propionate, ethyl alcohol, methylparaben, ethylparaben, propylparaben, butylparaben are suitable preservatives. Ethylenediaminetetraacetic acid (EDTA) may be used as a stabilizer and may also possess some antimicrobial activity that is synergistic with other antimicrobials.

These formulations are alcohol-free to avoid complications from using alcohol in oral dosage units intended for human use.

The pharmaceutical formulation described herein may comprise additional components that may include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; pharmaceutically acceptable polymeric or hydrophobic materials as well as other components.

The formulation may also comprise organoleptic components, which impart desirable sensory characteristics including taste, color, and smell. The organoleptic component may comprise a high intensity sweetener that improves sensory appeal such as sucralose liquid concentrate. These components may also include coloring agents that provide desired shades consistent with berry or cherry flavor products such as FD&C Yellow #6 or FD&C Red #40 from about 0.0025% to about 0.0075%. Flavoring agents such as cherry flavor or a concentration of berry flavor, and taste masking agents may be included to obscure the bitter flavor of active agents. The inventive formulations have attractive appearance, suitable texture and organoleptic properties. The components are mutually compatible in that they do not interfere with the bioactivity of the pharmaceutical agent or physical properties of the vehicle, and the components do not separate and retain their properties.

Mutual compatibility of the components means that they do not separate in preparation and storage for the equivalent of two years at room temperature (as indicated by three months accelerated stability testing at 40° centigrade and 75% relative humidity). Storage stability means that the materials do not lose their desirable properties during storage for the same period. Preferred compositions do not exhibit a drop in viscosity of more than 50% or an increase in viscosity of more than 100% during that period.

Pharmaceutical compositions that are useful in the methods of the invention may be prepared, packaged, or sold in formulations suitable for oral, parenteral and topical administration. Other contemplated formulations include nanoparticles, liposomal preparations, resealed erythrocytes containing the active ingredient, and immunologically-based formulations. The active ingredient may be contained in a formulation that provides quick release, sustained release or delayed release after administration to the patient.

The formulations of the pharmaceutical compositions described herein may be prepared by any method known or hereafter developed. In general, preparation includes bringing the active ingredient into association with a carrier or one or more other additional components, and then, if necessary or desirable, shaping or packaging the product into a desired single- or multi-dose unit. Specifically, the examples prepared for this application were prepared, in general, by dispersing the gelling agents in the vehicle phase, water was added gradually until sufficient hydration was achieved and then the active ingredients (or sub-phases thereof) were added. All other excipients (sweeteners, flavors, color, acid, anti-microbials) were then added.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient in each unit dose is generally equal to the total amount of the active ingredient which would be administered or a convenient fraction of a total dosage amount such as, for example, one-half or one-third of such a dosage. Additionally, kits of the novel spill-resistant formulations may be prepared. The kits may comprise two components. Component A would be an active ingredient or a combination of active ingredients in dry or liquid form, and component B would be comprised of all the ingredients of a spill-resistant formulation without the active ingredients. A dispenser or a consumer, immediately before consumption of the pharmaceutical composition, would mix the two components.

Although the descriptions of pharmaceutical compositions provided herein are principally directed to pharmaceutical compositions which are suitable for administration to humans, it will be understood by the skilled artisan, based on this disclosure, that such compositions are generally suitable for administration to any mammal. Preparation of compositions suitable for administration to various animals is well understood, and the ordinarily skilled veterinary pharmacologist can design and perform such modifications with routine experimentation based on pharmaceutical compositions for administration to humans.

### EXAMPLES

### Example 1

### Study Using Different Gelline Agents

Initial screening was done to examine the physical properties of the hydrocolloid polymers in solutions of glycerin and water. Sodium Alginate, 1.5% (Nelanders International, Mississauga Ontario), 2.0% Propylene Glycol alginate, 2.0% (Colloid 602—(Nelanders International, Mississauga Ontario), Kappa Gelcarin GP 911, 2.0%, Kappa (Gelcarin GP 812), 2.0%, and Xanthan gum (Tixacan 200), 1.0% (FMC Corporation, Philadelphia, Pa.) were dispersed in a 50:50 blend of glycerin/water. None of the hydrocolloid polymers, as tested, had spill-resistant characteristics. The results are shown in Table 1.

TABLE 1
Results and Observations

| Gelling Agent | Physical Description | Viscosity | Comments |
|---|---|---|---|
| Sodium alginate, 1.5% | Almost clear stringy soft | 7850 | Good spreading |
| (Colloid T-488) | gel | | |
| Propylene Glycol | Translucent soft, stringy | 12,000 | Slightly stiff spreading |
| alginate, 1.5 | | | |
| (Colloid 602) | | | |
| Carrageenan, Kappa 2.0% | Opaque, tan colored, pasty | 15,000 | Stiff, poor spreading, |
| (Gelcarin GP 911) | appearance | | tends to heap |
| Carrageenan, Kappa 2.0% | Opaque, tan colored, | 13,700 | Less stiff |
| (Gelcarin GP 812) | thinner appearance | | |
| Xanthan Gum, 1.0% | Translucent soft, stringy | 4,800 | Flows well, needs |
| (Tixacan 200) | | | stiffness |

### Example 2

### Test of Synergistic Effect

Experiments were done adding Xanthan gum to solutions of sodium alginate, carragenan, locust bean gum, guar gum, hydroxypropyl methylcellulose and sodium carboxymethylcellulose. The solutions were prepared in the same manner as described in Example 1 above. The results demonstrate that xanthan gum and locust bean gum have a synergistic effect with some, but not all, of the gelling agents tested (see Table II). The positive synergism allowed for a desired viscosity, with less gelling agent. However, the xanthan gum and locust bean gum also appeared to contribute to an undesirable stringy, elastic texture.

TABLE 2
Synergistic effects of Xanthan Gum

| Gelling agents | Description | Comments |
|---|---|---|
| Sodium alginate 1.5% | Translucent cohesive, | Requires stiffer, less stringy texture |
| Xanthan gum 0.5% | stringy | for spill-resistant characteristics |
| Sodium alginate 1.8% | Translucent cohesive, | More investigation needed, requires |
| Xanthan gum 0.3% | less stringy | less stringy texture for spill-resistant |
| | | characteristics |
| Carrageenan 2.0% | Opaque, gummy, | Requires less gummy, stringy texture |
| Xanthan gum 0.5% | stringy | for spill-resistant characteristics |
| Locust bean gum 0.12% | Opaque soft gel, | Requires more elastic, less stringy |
| Xanthan gum 0.54% | stringy | texture for spill-resistant |
| | | characteristics |
| Guar Gum 0.5% | Soft gel, less stringy, | More investigation needed, still too |
| Xanthan Gum 0.5% | good clarity | stringy for spill-resistant |
| | | characteristics |
| Hydroxy propyl | Very firm gel, less | More soft and flexible gel needed |
| methylcellulose 3.0% | flow, opaque milky | with better rheology for spill-resistant |
| Xanthan Gum 0.6% | color. | characteristics |
| Sodium carboxymethyl | The gel is clear, seems | Require less elastic, smoother texture |
| cellulose 1.0% | very stretchy, granular | for spill-resistant characteristics |
| Xanthan Gum 0.3% | texture. | |

### Example 3

### Carrageenan

Carrageenans added alone to the water/glycerin mixture resulted in a stiff non-flowing composition. (Table I). However, combination of 1.5% Propylene glycol alginate with 0.5% carrageenan provided a rheology that approximated the desired spill resistant characteristics. Carrageenan together with locust bean gum and xanthan provided made for a less stringy gel than locust bean gum and xanthan gum alone. The carrageenan/locust bean gum/xanthan gum mixture also spread smoothly when dispensed onto a spoon. The addition of guar gum to the carrageenan/locust bean gum/xanthan gum mixture did not appear to provide any further spill-resistant characteristics to the formulation.

TABLE 3
Effect of Carrageenan

| Gelling agents | Physical properties | Comments |
|---|---|---|
| Propylene glycol | Opaque, less stringy | Approximate |
| alginate 1.5% | | spill-resistance |
| Carrageenan 0.5% | | |
| Locust bean gum 0.10% | Opaque soft gel | Good spreading and |
| Xanthan gum 0.50% | | spill-resistance |
| Carrageenan 0.30% | | |
| Locust Bean Gum 0.1% | Flows well, weak gel, | Fails spill- |
| Carrageenan 1.0% | good clarity | resistant testing |
| Xanthan Gum 0.3% | | |
| Guar Gum 0.3% | | |
| Carrageenan 1.0% | Non gelling, liquid, | Fails spill- |
| Guar Gum 0.5% | light brownish color. | resistant testing |
| Carrageenan 0.6% | Very thin gel, flows | Good spreading |
| Hydroxypropylmethyl- | well, opaque, brownish | but fails |
| cellulose (Methocel) | color, Granular texture | inversion test. |
| 2.4% | | |

### Example 4

### Pseudoephedrine—Hydrocolloidal Polymers Spill-resistant Formulation.

A pseudoephedrine 0.27%/hydrocolloidal polymer mixture (0.375% carrageenan/0.625% xanthan gum/0.125% locust bean gum) composition was prepared in the following manner. Carrageenan (1.875 grams), locust bean gum (0.625 grams) and xanthan gum (3.125 grams) were added to 500 grams of glycerin in a water and heated to about 60° C.-70° C. for one hour while mixing. The mixture was cooled to about 40° C.-50° C. Butylparaben (0.25 grams) was dissolved in propylene glycol and added to the cooled mixture.

In a separate container, pseudoephedrine (2.70 grams) was dissolved in 10 mL of water and then added to the cooled mixture. Citric acid, sucrolose, colors and water were added. The pH of the final solution was 3.20 and the viscosity was 9400 cps. The formulation was determined to have acceptable spill-resistant characteristics, i.e. they spread well into a spoon, and remained on a spoon for greater than 60 seconds when the Spoon was shaken and inverted.

TABLE 4
Pseudoephedrine Formulation

| Ingredient | Weight (grams) | Weight (%) |
|---|---|---|
| Glycerin | 250 | 50.0 |
| Carra eenan | 1.875 | 0.375 |
| Locust Bean Gum | 0.625 | 0.125 |
| Xanthan Gum | 3.125 | 0.625 |
| Water purified | 228.6 | 45.72 |
| Citric Acid | 1.5 | 0.3 |
| Sucralose Liquid Concentrate | 1.0 | 0.20 |
| Propylene Glycol | 10.0 | 2.00 |
| Bu l araben | 0.25 | 0.05 |
| Pseudoe hedrine HCl | 1.35 | 0.27 |
| Grape Flavor | 0.45 | 0.09 |
| Bubble Gum Flavor | 0.20 | 0.04 |
| FD&C Red #40 1% solution | 0.70 | 0.14 |
| FD&C Blue #1 1% solution | 0.30 | 0.06 |
| TOTAL | 500.0 | 100.0 |

### Example 5

### Tragacanth and Locust Bean Gum

Tragacanth admixed with differing amounts of locust bean gum were tested for spill-resistant properties. Glycerin was evaluated as the co-solvent.

TABLE 5
Tragacanth

| Gelling agents | pH | Comments |
|---|---|---|
| Tragacanth 2% | NA | Translucent soft smooth gel, |
| | | runny flow |
| Tragacanth 2%/ | NA | Translucent soft smooth gel |
| 0.2% Locust Bean Gum | | |
| Tragacanth 2%/ | NA | Translucent smooth spreading |
| 0.4% Locust Bean Gum | | gel |
| Tragacanth 2%/ | 5.15 | Translucent, light yellow, |
| 0.4% Locust Bean Gum/ | | smooth gel with high yield |
| 50% glycerin | | value, low flow properties |

### Example 6

Method of preparing Spill-resistant composition of prednisolone acetate based on Locust Bean gum and Tragacanth gum.

8 grams of Locust Bean gum (AEP Colloids) and 32 grams of Tragacanth gum USP (Red Carnation brand, LV Lomas) were dispersed in 1000 mLs of glycerin using a propeller type mixer at about 500 rpm. 740 g water was added to the mixture and then heated the mixture was heated to 65° C. with slow stirring for 10 minutes. The mixture was allowed to cool to room temperature while maintaining slow agitation. Butylparaben (NIPA Laboratories) 6.0 g was dissolved in 200 g propylene glycol, then Prednisolone Acetate (Grand Pacific Co) 1.94 grams was added and dispersed using a propeller stirrer at about 300 rpm. And then added to the batch while mixing at about 300 rpm. Sucralose (McNeil Specialty Products) 4.0 g and Cherry flavor were added in the final mixing step.

Samples 1-4 of Prednisolone Acetate solution, as described in Table 6 below, were prepared using varying amounts of gum tragacanth and locust bean gum, with additional excipients. The samples all demonstrated, on spoon spreading, tilting, shaking and inversion tests, acceptable spill-resistant properties.

TABLE 6
Novel spill-resistant prednisolone acetate formulations

| | % weight/weight | | | |
|---|---|---|---|---|
| Ingredients | Sample 1 | Sample 2 | Sample 3 | Sample 4 |
| Purified Water | 47.44 | 42.93 | 37.64 | 37.05 |
| Sucralose Liquid | 0.20 | 0.20 | 0.20 | 0.20 |
| Concentrate | | | | |
| Gum Tragacanth | 1.50 | 1.00 | 1.30 | 1.60 |
| Locust Bean Gum | 0.40 | 0.40 | 0.40 | 0.40 |
| Propylene Glycol | 5.00 | 5.00 | 10.00 | 10.00 |
| Prednisolone Acetate | 0.097 | 0.097 | 0.097 | 0.097 |
| Butylparaben | 0.02 | 0.02 | 0.02 | 0.03 |
| Glycerin | 45.00 | 50.00 | 50.00 | 50.00 |
| Masking Agent | 0.20 | 0.20 | 0.20 | 0.20 |
| Cherry Flavor | 0.15 | 0.15 | 0.15 | 0.15 |
| pH | 5.31 | 5.05 | 4.92 | 4.76 |
| Viscosity | 15250 | 7720 | 8870 | 12300 |

We claim:

1. A composition comprising two or more hydrocolloidal polymers and an effective amount of a pharmaceutically active agent having:
a) a viscometric yield value of a semi-solid;
b) a spill-resistant consistency permitting the formulation to be squeezed by light manual pressure through a channel of about 1 to about 5 mm to spread in a spoon bowl sufficiently quickly for accurate measurement, and to remain in the spoon bowl without spilling for at least about one second and less than about 20 seconds on spoon tilting and for at least about 30 seconds upon spoon vibration; and
c) a pH of between about 2 to about 8.

2. The composition of claim 1 wherein the hydrocolloidal polymers are selected from the group consisting of carrageenan, alginates, propylene glycol alginate, xanthan gum, guar gum, locust bean gum, tragcanth, gum karaya, gum ghatti, gum arabic, agar agar, konjac, pectin, tara gum, gellan gum, pullulan, curdlan, gelatin, chitosan and any combination mixtures thereof.

3. The composition of claim 1 wherein the pharmaceutically active agent is selected from the group consisting of analgesics, anti-inflammatories, antipyretics, antibiotics, antimicrobials, laxatives, anorexics, antihistamines, antiastmatics, antidiuretics, antiflatuents, antimigraine agents, antispasmodics, sedatives, antihyperactives, antihypertensives, tranquilizers, decongestants, beta blockers; peptides, proteins, oligonucleotides and other substances of biological origin, and combinations thereof.

4. The composition of claim 1 wherein the pharmaceutically active agent is selected from the group consisting of acyclovir, atenolol, atropine, ciprofloxacin, dilitiazem, diphenhydramine, diphenhydramine HCl, epinephrine, azithromycin, clarithromycin, guaifenesin, ephedrine, glucosamine, glucosamine sulfate, hydrochlorothiazide, metoprolol, nortriptyline, phenytoin, propoxyphene, propranolol, terfenadine, tetracycline, pseudoephedrine, captopril, diclofenac, enalapril, furosemide, ketoprofen, phenobarbital, naproxen, ibuprofen, lovstatin, penicillin G, piroxicam and ranitidine and mixtures and salts thereof.

5. The composition of claim 1 having a viscosity of between about 5000 and about 15,000 cps.

6. The composition of claim 1, wherein the composition further comprises antimicrobial preservatives.

7. The composition of claim 1 further comprising a liquid base selected from the group consisting of propylene glycol, glycerin or sorbitol.

8. A method of preparing the composition of claim 1 involving the steps of
a. dispersing the gelling agents in the vehicle phase;
b. heating and mixing the dispersion of step a;
c. cooling step b;
d. dissolving at least one antimicrobial into the mixture of step b;
e. dissolving the active pharmacological ingredient in water, and adding to the mixture of step d;
f. adding all remaining excipients to step e.

9. A method of treating a patient in need of a therapeutically effective amount of a pharmaceutically active ingredient comprising administering the composition of claim 1.
